(19) United States
(12) Patent Application Publication
(10) Pub. No.: US 2010/0151529 A1
(43) Pub. Date: Jun. 17, 2010

(54) ENGINEERED PHOSPHITE DEHYDROGENASE MUTANTS

(75) Inventors: Huimin Zhao, Champaign, IL (US); Michael McLachlan, Urbana, IL (US); Tyler Johannes, Stillwater, OK (US)

Correspondence Address: BARNES & THORNBURG LLP, P.O. BOX 2786, CHICAGO, IL, 60690-2786, US

(73) Assignee: BIOTECHNOLOGY RESEARCH AND DEVELOPMENT CORPORATION, Peoria, IL (US)

(21) Appl. No.: 12/595,807

(22) PCT Filed: Apr. 18, 2008
(86) PCT No.: PCT/US08/60814

§ 371 Date: Oct. 13, 2009

Related U.S. Application Data
(60) Provisional application No. 61/000,758, filed on Apr. 19, 2007.

(51) Int. Cl. C12P 19/36; C12N 9/04; C07H 21/04; C12N 15/63

(52) U.S. Cl. 435/90; 435/190; 536/23.2; 435/320.1

(57) ABSTRACT

Phosphite dehydrogenase mutant enzymes provide relaxed cofactor specificity, increased thermostability, increased activity, solubility, and expression over the wild-type enzyme. The mutant enzymes are useful for nicotinamide cofactor regeneration.

### BACKGROUND

Biocatalysts are an attractive alternative to chemical catalysts in industry for many reasons, including high substrate specificity, an ability to operate under mild environmental conditions, and production of stereo-specific products. Enzymes such as oxidoreductases, however, often require cofactors such as NAD+/NADH or NADP+/NADPH which are oxidized or reduced during the reaction. Cofactor regeneration is an important consideration for the economical use of such enzymes in industrial processes as they are too expensive to be added stoichiometrically. One method that has found success is the coupling of the desired process to another enzyme reaction that converts the cofactor back to the required oxidation state. The most widely used enzyme for this coupling is the formate dehydrogenase from Candida boidinii.

However, the more recently discovered Pseudomonas stutzeri phosphite dehydrogenase (PTDH) that catalyzes NAD-dependent oxidation of phosphite into phosphate has several advantages over the formate dehydrogenase. These advantages include an inexpensive sacrificial phosphite substrate, a benign phosphate product, and a favorable equilibrium constant. Because natural enzymes are seldom optimal for use in industrial processes, PTDH is engineered to improve its catalytic properties.

The primary cost for regenerative biocatalytic processes in addition to cofactors, resides in the biocatalysts themselves. Therefore, in order to make a process economically viable, the regenerative enzyme must be relatively inexpensive in terms of cost per unit, making optimization of enzyme production and stability important. Wild type (WT) PTDH can be heterologously expressed in reasonable yields in E. coli, but improved expression levels would have important economic benefits. Furthermore, although the wild type enzyme is stable at 4° C., it undergoes fairly rapid inactivation under relatively mild temperatures.

### SUMMARY

Rational design based on a homology model of PTDH and directed evolution is used to greatly enhance the enzyme's thermostability. Directed evolution is also applied to significantly increase the solubility and turnover number of the PTDH enzyme. A saturation mutagenesis approach at thermostabilizing sites identified by error-prone PCR is useful. Using this approach also provides greater insight into the mechanism of thermal stabilization by analyzing multiple mutations at a particular site. The present disclosure provides mutations that increase the thermostability of the wild-type PTDH several fold. The approaches described herein are more useful and less time-consuming because they include an initial random mutagenesis screen followed by site directed saturation mutagenesis.

Saturation mutagenesis of phosphite dehydrogenase identified mutations that improved thermal stability compared to wild-type phosphite dehydrogenase. Error-prone PCR and saturation mutagenesis also generated thermostabilizing mutations. Some of the thermostabilizing mutations were context-dependent. Combination of thermostabilizing mutations at each site resulted in a PTDH variant that showed a 100-fold increase in half-life of thermal inactivation at 62° C. over a parent 12×PTDH mutant.

One or more amino acid mutations in wild-type phosphite dehydrogenase improved protein solubility, enzyme activity, relaxed specificity for nicotinamide cofactors, and thermostability. Engineered mutant phosphite dehydrogenases disclosed herein are useful in regenerating NADH, NADPH and also in the production of various products of commercial interest that require NADH and NADPH regeneration.

A mutant phosphite dehydrogenase (PTDH) with an increased thermostability and relaxed cofactor specificity for nicotinamade cofactor regeneration as compared to a wild-type phosphite dehydrogenase includes a mutation selected from a group that includes Q132K, Q137H, R275L, L276C, A146S, F198M, and T101A.

A mutant phosphite dehydrogenase (PTDH) that includes mutations designated as Q132K, Q137H, R275L, and L276C compared to the wild-type PTDH.

A mutant phosphite dehydrogenase (PTDH) that includes mutations designated as Q132K, Q137H, R275L, L276C and A146S compared to the wild-type PTDH.

A mutant phosphite dehydrogenase (PTDH) that includes mutations designated as Q132K, Q137H, R275L, L276C, A146S, and F198M compared to the wild-type PTDH.

A mutant phosphite dehydrogenase (PTDH) (“Opt12”) that includes mutations designated as Q132K, Q137H, R275L, L276C, D13E, M26I, E175A, E332N, C336D, I150F, Q215L, A319E, V315A, V71I, E130K, I313L, and A325V compared to the wild-type PTDH.

A mutant phosphite dehydrogenase (PTDH) (“Opt13”) that includes mutations designated as Q132K, Q137H, R275L, L276C, A146S, D13E, M26I, E175A, E332N, C336D, I150F, Q215L, A319E, V315A, V71I, E130K, I313L, and A325V compared to the wild-type PTDH.

A mutant phosphite dehydrogenase (PTDH) (“Opt14”) that includes mutations designated as Q132K, Q137H, R275L, L276C, A146S, F198M, D13E, M261, E175A, E332N, C336D, I150F, Q215L, A319E, V315A, V71I, E130K, I313L, and A325V compared to the wild-type PTDH.

A nucleic acid molecule encoding any one of the phosphite dehydrogenase mutants disclosed herein.

A phosphite dehydrogenase mutant disclosed herein is substantially purified, for example about 90% pure, or about 95% pure, or about 99% pure. The mutant phosphite dehydrogenases include recombinant, heterologously expressed forms of phosphite dehydrogenases.

A phosphite dehydrogenase mutant that includes an amino acid mutation designated A176R in combination with Opt12 or Opt13 mutations.

A method of generating at least one of NADH and NADPH includes the steps of:

(a) providing a mutant phosphite dehydrogenase, wherein the mutant has an amino acid mutation selected from the group consisting of mutations Q132K, Q137H, R275L, L276C, A146S, F198M, and T101A as compared to the wild-type. and;

(b) generating at least one of NADH and NADPH by a reduction reaction of at least one of NAD+ and NADP+.

Use of a phosphite dehydrogenase disclosed herein to regenerate one of NAD+, NADP+ or both NAD+ and NADP+.

“Improved characteristic” refers to a statistically significant, measurable increase in a characteristic, or an improvement in at least one feature such as kinetics, thermostability, solubility, relaxed specificity in a mutant phosphite dehydrogenase as compared to a wild-type phosphite dehydrogenase.

“Mutation” refers to a change or alteration at the amino acid or at the nucleotide level including insertion, deletion, and substitution of amino acids or nucleotides. “Mutant” refers to a protein or a peptide or a nucleic acid that is different either structurally or functionally from the wild-type counterpart.

A suitable host cell includes for example, bacteria, yeast, and plants. Suitable bacteria includes E. coli.

### BRIEF DESCRIPTION OF THE DRAWINGS

FIG. 1 shows an amino acid sequence of wild-type PTDH (SEQ ID NO: 1).

FIG. 2 shows optimal temperatures of stabilized phosphite dehydrogenase mutants.

FIG. 3 shows a homology model of the Opt14 mutant of phosphite dehydrogenase including the fourteen residues involved in improving thermal stability.

### DETAILED DESCRIPTION

Amino acid changes or mutations were introduced in the wild-type phosphite dehydrogenase (WT PTDH) (SEQ ID NO: 1) from Pseudomonas stutzeri to yield a plurality of phosphite dehydrogenase mutants with various improved characteristics. Phosphite dehydrogenases from other sources are also suitable to the extent they share structural similarity and/or functional homology. Some of the mutations and their properties are disclosed in Table 4. Phosphite dehydrogenase mutants disclosed herein have one or more of the following characteristics:
(a) higher catalytic rate (k_{cat});
(b) increased efficiency (k_{cat}/Kₘ);
(c) higher thermostability;
(d) relaxed cofactor specificity (both the natural cofactor NAD⁺ and cofactor NADP⁴);
(e) increased solubility; and
(f) increased expression

Error-prone PCR was performed on the 12×PTDH mutant, generated previously by three previous rounds of error-prone PCR and high throughput screening. This error-prone PCR screening produced the variants 4-4G2 and 4-11C3. DNA sequencing revealed two new mutations, A146S and F198I. The mutation A146S was cloned into the parent PTDH template in pET15b, expressed and purified. The resultant PTDH mutant is designated as “12X+A146S” variant. The parent PTDH already contained five mutations that increased solubility and activity: D13E, M26I, E175A, E332N, and C336D. The parent's thermostability was almost identical to that of the wild type enzyme. The A146S mutation was shown to increase the half-life of thermal inactivation at 45° C. from around 1 minute, to 8 minutes (Table 1). The F198I mutation led to low activity and was not further cloned into the parent background.

Saturation mutagenesis was performed separately on each of the following residues in the parent PTDH template: V71, E130, Q132, Q137, I150, Q215, R275, L276, I313, V315, A319, and A325. Residues A146 and F198 were mutated in the context of the 12×PTDH mutant. The libraries were screened for increased thermostability at 45° C. for the parent PTDH template, or 62° C. for the 12×PTDH template, and promising variants were selected for further analysis. Variants that showed increased stability were sequenced to identify the mutations. These variants were sub-cloned into the vector pET15b followed by protein purification for characterization. Table 1 shows the half-lives of thermal inactivation of the mutant proteins in the parent template when incubated at 45° C. Apart from the mutations known from error-prone PCR of this protein, no additional substitutions conferring increased stability were found for residues V71, A146, I150, I313, V315, A319, or A325. For residue E130, glutamine and arginine substitutions increased stability substantially. A lysine substitution at residue Q132 showed slightly higher stability than that of the known arginine substitution, as did a histidine substitution at residue Q137 compared to the arginine substitution. The methionine substitution increased the stability when present at residue F198, and when at residue Q215 gave a moderate increase to stability but to a lesser extent than the known leucine mutation. An arginine to leucine substitution at residue R275 greatly increased stability. Many new beneficial mutations were seen at residue L276, namely histidine, serine, arginine, and cysteine.

During screening of the residue A319 saturation library, a spontaneous threonine to alanine mutation was observed at position 101. When T101A was introduced separately into the parent enzyme, it conferred a fourfold increase in stability. In the context of the 12×PTDH variant, however, this mutation led to a decrease in stability.

The most thermostabilizing mutation discovered for each particular site was incorporated into the 12×PTDH mutant. This was performed for K₁₃₂, H137, L275, and C276, forming an optimized thermally stable phosphite dehydrogenase termed “Opt12”. The addition of A146S to Opt12 led to the “Opt13” variant, and the further addition of F198M led to Opt14, the final mutant showing 14 amino acid substitutions from the parent enzyme, and 19 amino acid substitutions from the wild type enzyme.

Effectiveness of saturation mutagenesis is demonstrated herein. This identification of novel mutations successfully demonstrated the usefulness of including saturation mutagenesis in a directed evolution strategy by further improving the stability of phosphite dehydrogenase by 100 fold at 62° C. The thermostability of the 12× phosphite dehydrogenase was improved by altering the amino acid at sites previously identified by error-prone PCR to be involved in stability. At eight of the 12 original sites, no better mutations were discovered, but for sites 132, 137, 275, and 276, new thermostabilizing amino acid substitutions were revealed. The results showed that the thermostabilizing sites were not equally conducive to modification, with residue L276 showing five substitutions that were more stable, whereas residues such as V71 or I150 yielded no other thermostabilizing mutations. The number of base changes found was one for Q132K, one for Q137H, two for A146S, two for F198M, two for R275L (although the minimum needed was one), and three for L276C. Saturation mutagenesis thus focused screening at the examined sites, avoiding the bias of error-prone PCR and allowing the discovery of amino acid substitutions requiring multiple changes in a single codon that would be very rare by error-prone PCR.

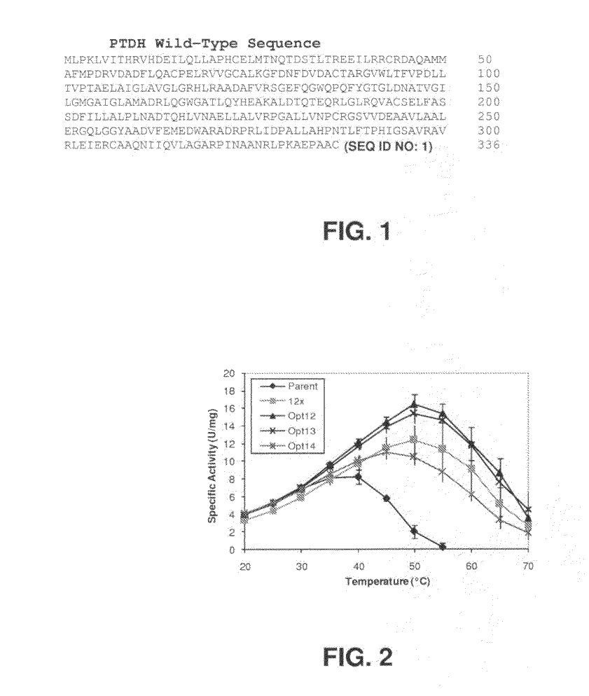

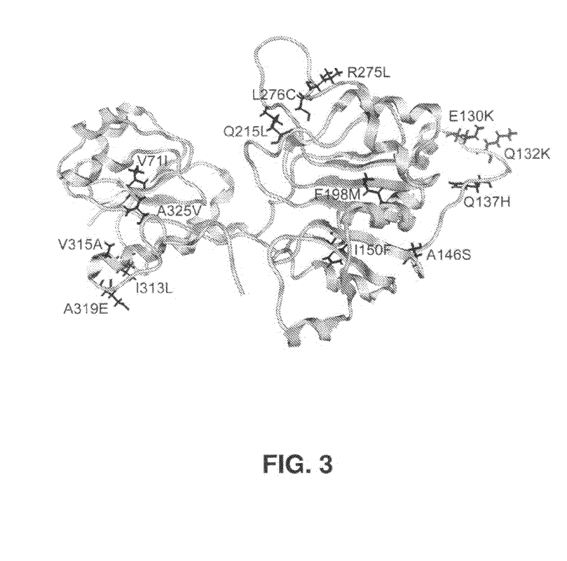

Two improved mutants, designated as Opt13 and Opt14 showed a trade-off between activity and stability. The most thermally stable variant was Opt14, as indicated by the two fold increase in half-life at 62° C. However at 25° C., the k_{cat}/K_{M} indicates that Opt13 is the more efficient enzyme, and from FIG. 1 it can be seen that Opt13 shows a higher activity at elevated temperatures. Therefore, the choice of a variant to use may depend on the conditions of the reaction.

The decrease in optimal temperature for Opt14 was unexpected. Typically, an increase in stability would be accompanied by an increase rather than a decrease in temperature optimum. These effects illustrate that thermostability and thermoactivity are distinguishable features of an enzyme.

Protein stability is influenced by multiple factors including hydrogen bonding networks, hydrophobic interactions, entropic effects, packing efficiency, multimerization, and amino acid composition. Mutations can be introduced to exploit these factors, however there is no general method one can use to predict which changes should be made to increase the stability of a given protein. Rational approaches can be attempted, or one can use random mutagenesis and screening in a directed evolution strategy. By incorporating saturation mutagenesis here, further insights were gained into how the sites modified in the context of the 12× mutant influenced stability.

For the buried residues V71 and I150, no other stabilizing mutations were observed, and the mechanism of thermostabilization at these sites is still expected to be related to hydrophobic interactions. Residues I313 and V315 are within an alpha helix and no further mutations were found for these sites, leaving us with the same suggested mechanism of alpha helix stabilization. Residues A319 and A325 are in an unstructured region near the C-terminus and may help anchor this region. The A319E mutation allows for hydrogen bonding between the carboxyl of glutamate and the amino group of glutamine 314. Residue Q215 is surface exposed, but when mutated from the hydrophilic glutamine residue to either leucine or methionine, both more hydrophobic, the stability is increased. This likely indicates that hydrophobic interactions with surrounding amino acids are generated by these mutations.

Residues E130, Q132, and Q137 are in the loop between α6/β5, close to residues R275 and L276 on the other subunit of the dimer, and interactions involving some of these sites may contribute to dimer stabilization. The negatively charged E130 could be more stably replaced by the positively charged lysine or arginine, or the neutral glutamine. This along with the negatively charged residues close to E130 on the other subunit (E264, E266, D267, and D272) would support a mechanism of balancing charge in the area. The enzyme was more stable when Q132 was replaced by the positively charged lysine or arginine, and when Q137 was replaced by positive arginine or the neutral/positive histidine. Some of the stabilization may arise due to the removal of glutamine 132/137 since the residue can lead to protein denaturation by deamidation, especially when the next residue is small such as G133. Mutagenesis of R275 showed that leucine is even more stable than the previously found glutamine, both of which are neutral residues which may influence the charge distribution in the area beneficially. The many stabilizing mutations at residue L276 are polar or positively charged, and more hydrophilic than the parent leucine. They may introduce hydrogen bonds with water molecules or other residues to increase the stability.

Residue A146 is positioned at the beginning of β5 after an unstructured region, with backbone hydrogen bonds between its carboxyl group and the amino group of T170, and its amino and the carboxyl group of L143. Replacing the alanine with a serine would preserve these bonds but also allow the serine hydroxyl to hydrogen bond with the backbone of G142 or L143, thus helping to anchor the unstructured region. The final mutation, F198M, is situated on an alpha helix in a hydrophobic area formed by a beta sheet. Methionine is more stabilizing to an alpha helix than phenylalanine, and while being less hydrophobic, it is more flexible which may allow it to fill the space better.

The majority of the mutations have an additive effect. When introduced separately, the mutations I313L, V315A, and A325V were not stabilizing in the parent sequence. The data provided herein has demonstrated the benefit of applying saturation mutagenesis to improve protein stability and to decipher the mechanisms of thermal stabilization. By accessing all possible amino acids at these thermostabilizing sites, several mutations were found to increase the stability beyond those initially identified. The further engineering of the 12× mutant resulted in two PTDH variants, Opt13 and Opt14, with significantly enhanced stability at high temperatures without compromising turnover numbers, which is useful for cofactor regeneration applications.

Amino acid mutations identified for the P. stutzeri PTDH disclosed herein are used as templates or foundations for identifying corresponding mutations in PTDH enzymes derived from other sources. For example, through homology modelling methods disclosed herein, structurally and functionally conserved domains are delineated among various PTDH enzymes. Then, relevant mutations disclosed herein can be engineered using site-directed mutagenesis or any suitable method.

The combinations of a plurality of mutations cannot be simply predicted to function as did the individual mutations because of the underlying structural and functional differences that result from the mutations. Present understanding of protein structure and function alone does not yet guarantee that rationally designed changes will yield the predicted outcomes. In fact, protein engineers frequently have been surprised by the range of effects brought about by single mutations designed to change only one specific and simple property in a protein. As a result, possible changes to the protein sequences have been made by mutagenesis/recombination, and then the functionally improved variants were isolated by selection or screening. Further analysis of these variants revealed several of the improved characteristics disclosed herein. The effects of thermostabilizing mutations are not necessarily independent and cumulative and therefore one cannot predict with certainty that a plurality of the mutations can be combined without a loss of one or more of the properties, for example, engineering thermostable mutations into the mutants with improved activity without losing their thermostabilizing effects, requires inventive efforts.

The amino acid sequences of homologous phosphite dehydrogenases may differ from the amino acid sequences disclosed herein by an insertion or deletion of one or more amino acid residues and/or the substitution of one or more amino acid residues by different amino acid residues. Preferably, amino acid changes are of a minor nature, that is conservative amino acid substitutions that do not significantly affect the folding, thermostability, expression, and/or activity of the protein; small deletions, typically of one to about 30 amino acids; small amino- or carboxyl-terminal extensions, such as an amino-terminal methionine residue; a small linker peptide of up to about 20-25 residues; or a small extension that facilitates purification by changing net charge or another function, such as a poly-histidine tract, an antigenic epitope or a binding domain.

The term ‘consisting essentially’ as used herein refers to amino acid or a nucleic acid sequence that contains one or more of the mutations disclosed herein and any other sequence that does not substantially affect the improved characteristics of the mutant phosphite dehydrogenases disclosed. For example, the phosphite dehydrogenase may have a plurality of the disclosed mutations and any other amino acid substations, deletions, insertions without substantially affecting the functionality of the disclosed engineered phosphite dehydrogenases.

The term substantially purified refers to a preparation of mutant phosphite dehydrogenase that is at least about 90% pure or about 95% pure or about 99% pure.

The disclosures of commonly owned patent applications PCT/US06/00135 and U.S. Ser. No. 10/865,146 are incorporated herein by reference in their entirety, to the extent they disclose the various phosphite dehydrogenase mutants and uses thereof.

### EXAMPLES

The following examples are illustrative and do not limit the scope of the various methods and compositions disclosed herein.

### Example 1

### Engineering Phosphite Dehydrogenase Mutants with Improved Thermostability Through Saturation Mutagenesis

Overlap extension PCR was used to generate libraries of PTDH genes encoding all possible amino acids at sites 71, 130, 132, 137, 150, 215, 275, 276, 313, 315, 319, and 325. Saturation mutagenesis was performed separately on each of the following residues in the parent PTDH template: V71, E130, Q132, Q137, I150, Q215, R275, L276, I313, V315, A319, and A325. The parent construct was amplified as two fragments that overlapped around the site that was mutated. Fragment 1 used primers pRW2_For_NdeI (5′-TTT TTG GAT GGA GGA ATT CAT ATG-3′) and a site specific reverse primer. Fragment 2 used a site specific forward primer and PTDH_Rev_PciI (5′-GTA CGT CGA TAC ATG TTT ATC AGT CTG CGG CAG G-3′). PCR was performed in a volume of 50 μl with cycle conditions of 94° C. 4 min, (94° C. 45 s, 55° C. 45 s, 72° C. 45 s)×25 cycles, 72° C. 7 min. Fragments 1 and 2 were gel purified using QIAEX II Gel Extraction kit (Qiagen, Valencia, Calif.). The PCR products were digested with DpnI to remove the parent plasmid (3 hours at 37° C. with 10 U of DpnI), and purified with QIAquick PCR purification kit (QIAGEN). Fragments 1 and 2 (0.026 ng×length in base pairs) were joined by overlap extension to create the full-length gene. A 20 μl reaction with PfuTurbo DNA polymerase (Stratagene, La Jolla, Calif.) was cycled for 95° C. for 2 minutes, 10 cycles of 94° C. for 1 minute, 55° C. for 1 minute, and 72° C. for 3 minutes, with a final extension of 72° C. for 10 minutes. Four microliters of this reaction mixture was used as a template for a 100 μl PCR reaction using the primers pRW2_For_NdeI and PTDH_Rev_PciI. The reaction was purified with QIAEX II Gel Extraction kit (QIAGEN). The insert was digested with NdeI and PciI, and ligated into the pRW2 vector. This library was electroporated into E. coli BW25141 competent cells.

Library Screening: A 96 well plate assay was used to screen for phosphite dehydrogenase activity, as described in Johannes et al., (2005). Appl Environ Microb 71: 5728-5734. For screening in the parent genetic background, a temperature of 42° C. was used, while 62° C. was used for the libraries in the 12×PTDH mutant background.

Enzyme Purification: Selected mutants were cloned into pET15b as a N-terminal His-tagged construct and verified by DNA sequencing using the BigDye™ Terminator sequencing method and an ABI PRISM 3700 sequencer (Applied Biosystems, Foster City, Calif.). Small scale protein purification was carried out as described in Johannes et al. (2005). Glycerol was added to a concentration of 20% and the enzyme was stored at −80° C.

Enzyme Kinetics: Enzyme kinetics were determined at 25° C. by measuring the activity of 3 μg enzyme when either NAD⁺ or phosphite was held at 2 mM, and the other substrate was present at 5, 50, 100, 400, or 2000 μM. The data were used to calculate the kinetic constants by fitting of the Michaelis-Menten equation using Microcal Origin 5.0 (OriginLab Corporation, Northampton, Mass.).

Residues A146 and F198 were mutated in the context of the 12×PTDH mutant. The libraries were screened for increased thermostability at 45° C. for the parent PTDH template, or 62° C. for the 12×PTDH template, and promising variants were selected for further analysis. Variants that showed increased stability were sequenced to identify the mutations. These variants were sub-cloned into the vector pET15b followed by protein purification for characterization. Table 1 shows the half-lives of thermal inactivation of the mutant proteins in the parent template when incubated at 45° C. Apart from the mutations known from error-prone PCR of this protein (Johannes et al. 2005), no additional substitutions conferring increased stability were found for residues V71, A146, I150, I313, V315, A319, or A325. For residue E130, glutamine and arginine substitutions increased stability substantially. A lysine substitution at residue Q132 showed slightly higher stability than that of the known arginine substitution, as did a histidine substitution at residue Q137 compared to the arginine substitution. The methionine substitution increased the stability when present at residue F198, and when at residue Q215 resulted in a moderate increase to stability but to a lesser extent than the known leucine mutation. An arginine to leucine substitution at residue R275 greatly increased stability. Many new beneficial mutations were seen at residue L276, namely histidine, serine, arginine, and cysteine.

During screening of the residue A319 saturation library, a spontaneous threonine to alanine mutation was observed at position 101. When T101A was introduced separately into the parent enzyme, it conferred a fourfold increase in stability. In the context of the 12×PTDH variant, however, this mutation led to a decrease in stability.

### Example 2

### Thermostability and Optimal Temperature Determination

Purified enzyme was diluted to 0.2 mg/ml in 50 mM morpholinepropanesulfonic acid (MOPS) buffer (pH 7.25), incubated at 45° C., 50° C., or 62° C., and samples were removed at varying time points. Activity of each sample was measured by adding 10 μl of enzyme to 490 μl of 2 mM phosphite/1 mM NAD⁺ and the initial rate of increase in absorbance at 340 nm was monitored in a Cary 100 Bio UV-Visible spectrophotometer (Varian, Palo Alto, Calif.). The data was modeled with an exponential decay curve and the half-life determined from the exponential coefficient. The activity of improved enzyme variants was measured at temperatures between 20° C. and 70° C. in 5° C. increments.

Tₘ Measurement by Circular Dichroism: To measure the melting temperature (Tₘ) of the enzyme variants, thermal denaturation was monitored by circular dichroism. Samples were prepared by adding 120 μg of protein to 50 mM potassium phosphate buffer (pH 7.0)/1 M urea in a final volume of 2 ml. The sample was placed in a quartz cuvette with a 1 cm path-length and heated in a Peltier controlled cell at a rate of 1° C. per minute. Ellipticity was monitored at 222 nm in a Jasco spectropolarimeter (Jasco Inc, Easton, Md.). The midpoint of the denaturation curve was determined with Microcal Origin 5.0 software (Northampton, Mass.).

The most thermostabilizing mutation discovered for each particular site was incorporated into the 12×PTDH mutant. This was performed for K132, H137, L275, and C276, forming an optimized thermally stable phosphite dehydrogenase termed Opt12. The addition of A146S to Opt12 led to the Opt13 variant, and the further addition of F198M led to Opt14, the final mutant showing 14 amino acid substitutions from the parent enzyme, and 19 amino acid substitutions from the wild type enzyme.

### Example 3

### Identification of Two Novel Mutations that Increase Thermostability Through Error Prone PCR

Error-prone PCR was performed on the 12×PTDH mutant, which had been generated by three rounds of error-prone PCR and high throughput screening (Johannes et al. 2005). This produced the variants 4-4G2 and 4-11C3. DNA sequencing revealed two new mutations, A146S and F1981. The mutation A146S was cloned into the “parent” PTDH template in pET15b, expressed and purified. Note that the “parent” PTDH contained five mutations that increased solubility and activity: D13E, M26I, E175A, E332N, and C336D. Its thermostability was almost identical to that of the wild type enzyme (Johannes et al. 2005). The A146S mutation was shown to increase the half-life of thermal inactivation at 45° C. from around 1 minute, to 8 minutes (Table 1). The F198I mutation led to low activity and was not cloned into the parent background.

### Example 4

### Characterization of Thermostable Mutants

The half-lives of thermal inactivation at 45° C. for enzymes containing single mutations in the parent background are displayed in Table 1. The parent enzyme has a half-life at this temperature of around one minute. The wild-type amino acid sequence of a PTDH is shown in FIG. 1. The best single mutations increase this by over ten-fold. Interestingly, some of the mutations previously found did not show significant increases when introduced individually into the parent enzyme, namely I313L, V315A, A325V, and to a lesser extent V71I. These were all found in the second and third rounds of error-prone PCR (Johannes et al. 2005). Table 2 shows the half-lives of thermal inactivation of the optimal mutants at 45° C., 50° C., and 62° C. By using the best mutations at each of the 12 initial sites, improvements in half-life over the 12× mutant were seen by 1.5, 2.7, and 8.8 fold at 45, 50, and 62° C., respectively. The addition of thermostabilizing mutations at sites 146 and 198 led to a dramatic increase in stability at 62° C., with notable improvements at lower temperatures. The Opt14 mutant had a half-life of 450 minutes at 62° C., increased over 100-fold from the 12× mutant. At 45° C., the half-life of thermal inactivation of the Opt14 mutant was approximately doubled compared to that of the 12× mutant, representing over 23,000-fold improvement compared to the parent enzyme.

The apparent melting temperatures of all the PTDH mutants were determined by circular dichroism. Unfolding was seen to be irreversible, and the mid-points of the denaturation curves representing the melting temperature Tₘ are reported in Table 1 and Table 2. The Tₘ of the parent enzyme was just under 40° C., with single mutations having effects ranging from very little up to increasing Tₘ by 7° C. The 12× mutant had a Tₘ of around 60° C., and the three improved variants, Opt12, Opt13, and Opt14, were around 64° C.

The optimal temperature of the stabilized enzymes was examined by measuring the initial activity at temperatures ranging from 20° C. to 70° C., and is shown in FIG. 2. The parent phosphite dehydrogenase has an optimal temperature of around 40° C. The 12×, Opt12, and Opt13 mutants have an optimal temperature around 50° C., with the Opt14 optimum decreasing to 45° C. The activities of the Opt12 and Opt13 mutants were higher than those of the 12× mutant and the parent enzyme, while the Opt14 had activities lower than the 12× mutant at temperatures above 45° C.

The improved mutants from this work were subjected to kinetic analysis at 25° C. with respect to NAD⁺ and phosphite (Table 3). The Opt12 and Opt13 variants showed similar kinetics with a k_{cat} slightly higher than the 12× mutant but lower than the parent. The K_{M,NAD+} for Opt12 and Opt13 was intermediate between the parent and the 12× mutant, while the K_{M,Pt-H} was higher than both. The corresponding values of k_{cat}/K_{M,NAD+} of 4.3 and 4.0 μM⁻¹ min⁻¹ for Opt12 and Opt13, respectively, were similar to the parent, but lower than that of the 12× mutant (4.9 μM⁻¹ min⁻¹). The Opt14 mutant showed a similar k_{cat} but a doubled K_{M,NAD+} and tripled K_{M,Pt-H} relative to the 12× mutant, which led to a reduction in k_{cat}/K_{M}.

### Example 5

### Production of (R)-Phenylethanol Using the Opt 12, Opt13 and Opt 14 Mutant PTDH

Small-scale batch reactions containing 20 mM acetophenone is carried out using wild-type PTDH, the Opt 12, Opt13 and Opt 14 PTDH mutants, and commercially available NADP-specific FDH mutant (mut-Pse FDH). The time course of production of (R)-phenylethanol with NADPH regeneration is measured. The rate of reaction for the 12x+A176R mutant PTDH mutant is measured.

### Example 6

### Continuous Production of Xylitol Using the Opt 12, Opt13 and Opt 14 PTDH Mutants PTDH

The stability and effectiveness of the Opt 12, Opt13 and Opt 14 PTDH mutants is demonstrated in a continuously operated enzyme membrane reactor (EMR) along with xylose reductase (XR). The conversion of D-xylose to xylitol is chosen as a model to evaluate the performance of the PTDH/phosphite regeneration system. Several batch reactions are carried out to determine optimal reaction conditions for the reactor. The continuous production of xylitol is performed in a 10-mL stainless-steel reactor. The reactor is continuously operated for 180 hours and a substrate flow rate of 2.4 mL/h is used. Since there are no side reactions in the system described herein, yield and conversion are identical. The deactivation of the enzymes under these reactor conditions is approximately 2.8% per day. The conversion gradually decreases as time elapses due to this deactivation. The main reaction is efficiently coupled to the enzymatic regeneration of the cofactor.

TABLE 1
Mutations identified from saturation
mutagenesis and their half-lives of thermal
inactivation and melting temperatures. The
mutations with asterisks are original
mutations found in the 12x mutant.

| | | | t_{1/2} | Tm |
|---|---|---|---|---|
| Site | Mutant | Mutation | (min 45° C.) | (° C.) |
| | Parent | | 1.07 ± 0.07 | 39.7 ± 0.3 |
| 71 | V71I* | GTC→ATC | 1.30 ± 0.11 | 40.3 ± 0.1 |
| 101 | T101A | ACG→GCG | 4.52 ± 0.66 | 41.1 ± 1.6 |
| 130 | E130Q | GAG→CAG | 7.43 ± 0.25 | 47.0 ± 4.6 |
| 130 | E130R | GAG→CGG | 9.30 ± 0.43 | 46.1 ± 1.6 |
| 130 | E130K* | GAG→AAG | 12.56 ± 0.35 | 47.3 ± 2.0 |
| 132 | Q132K | CAG→AAG | 2.76 ± 0.01 | 41.5 ± 2.5 |
| 132 | Q132R* | CAG→CGG | 2.30 ± 0.01 | 39.0 ± 3.5 |
| 137 | Q137H | CAG→CAT | 4.62 ± 0.80 | 42.7 ± 1.0 |
| 137 | Q137R* | CAG→CGG | 3.90 ± 0.14 | 42.7 ± 2.4 |
| 146 | A146S | GCT→TCC | 8.23 ± 0.49 | 41.2 ± 1.6 |
| 150 | I150F* | ATC→TTC | 7.00 ± 1.60 | 42.0 ± 0.6 |
| 198 | F198M | TTC→ATG | 2.15 ± 0.13 | 40.6 ± 1.4 |
| 215 | Q215M | CAG→ATG | 2.46 ± 0.15 | 40.8 ± 1.4 |
| 215 | Q215L* | CAG→CTG | 8.70 ± 0.80 | 40.9 ± 1.8 |
| 275 | R275L | CGG→CTC | 9.09 ± 0.40 | 41.6 ± 0.6 |
| 275 | R275Q* | CGG→CAG | 4.60 ± 0.40 | 40.0 ± 1.0 |
| 276 | L276C | CTG→TGC | 11.72 ± 0.18 | 44.7 ± 1.0 |
| 276 | L276H | CTG→CAC | 2.05 ± 0.3 | 40.5 ± 0.4 |
| 276 | L276R | CTG→CGG | 7.76 ± 0.71 | 43.6 ± 2.4 |
| 276 | L276Q* | CTG→CAG | 3.58 ± 0.23 | 41.4 ± 0.3 |
| 276 | L276S | CTG→TCC | 3.29 ± 0.04 | 39.8 ± 0.6 |
| 313 | I313L* | ATC→CTC | 1.05 ± 0.03 | 39.2 ± 0.3 |
| 315 | V31SA* | GTA→GCA | 1.14 ± 0.11 | 40.7 ± 0.7 |
| | | GCG→GAG | | 41.9 ± 0.1 |
| 319 | A319E/T1O1A | ACG→GCG | 5.34 ± 0.35 | |
| 319 | A319E* | GCG→GAG | 2.14 ± 0.06 | 40.6 ± 0.6 |
| 325 | A325V* | GCG→GTG | 1.01 ± 0.03 | 39.3 ± 0.1 |

TABLE 2
Thermal stability of optimal mutants.

| | t_{1/2} | t_{1/2} | t_{1/2} | Tₘ |
|---|---|---|---|---|
| Enzyme | (min, 45° C.) | (min, 50° C.) | (min, 62° C.) | (° C.) |
| Parent | 1.07 ± .07 | nd | nd | 39.7 ± 0.3 |
| 12x | 13246 ± 3289 | 3868 ± 355 | 4.0 ± 0.8 | 59.7 ± 1.0 |
| Opt12 | 20441 ± 7536 | 10331 ± 1757 | 35 ± 1.2 | 63.9 ± 1.3 |
| Opt13 | 24875 ± 3770 | 14757 ± 1129 | 210 ± 21 | 64.2 ± 1.1 |
| Opt14 | 25518 ± 2144 | 15415 ± 1049 | 450 ± 49 | 64.4 ± 0.8 |

nd = not determined

TABLE 3
Enzyme kinetics for the thermostable mutants.

| | k_{cat} | K_{M} | K_{M} | k_{cat}/K_{M, NAD} |
|---|---|---|---|---|
| Enzyme | (min⁻¹) | (μM, NAD⁺) | (μM, Pt—H) | (μM⁻¹min⁻¹) |
| Parent | 262 ± 7 | 66 ± 7 | 57 ± 4 | 4.0 |
| 12x | 195 ± 4 | 40 ± 3 | 46 ± 6 | 4.9 |
| Opt12 | 213 ± 3 | 50 ± 5 | 79 ± 15 | 4.3 |
| Opt13 | 213 ± 3 | 54 ± 6 | 90 ± 17 | 4.0 |
| Opt14 | 219 ± 5 | 105 ± 15 | 142 ± 28 | 2.1 |

TABLE 4
List of various mutations, their designations, and some of their
properties for engineered phosphite dehydrogenase (PTDH) mutants.

| PROPERTY IMPROVED | MUTATIONS | PARAMETERS |
|---|---|---|
| Activity and Expression | D13E, M26I, E175A, | Designated the “parent” mutant. |
| | E332N, C336D | Soluble expression of PTDH in |
| | | E. coli was increased more than |
| | | 3-fold, while the rate of turnover |
| | | was increased about 2-fold, |
| | | effectively lowering the cost of |
| | | the enzyme by >6-fold. Large- |
| | | scale production of the soluble |
| | | expression PTDH mutant enzyme |
| | | by fermentation resulted in ~6- |
| | | times higher yield (Units/Liter) |
| | | than the WT PTDH. |
| Relaxed cofactor specificity | E175A, A176R | Designated the “double mutant”. |
| | | Increased efficiency for NAD⁺ |
| | | by approximately 4-fold while |
| | | increasing efficiency for NADP⁺ |
| | | approximately 1000-fold |
| | | compared to the wild-type |
| | | PTDH. |
| Thermostability | Q132R, Q137R, I150F, | Designated the “12X” mutant. |
| | Q215L, R275Q, L276Q, | T₅₀ is 20° C. higher and half-life of |
| | A319E, V315A, V71I, | thermal inactivation at 45° C. |
| | E130K, I313L A325V | is >7000-fold greater than that of |
| | | the “parent” PTDH. |
| Thermostability | K132, H137, L275, and | Opt12 |
| | C276 + 12X mutations | |
| Thermostability | A146S + Opt12 | Opt13 |
| Thermostability | F198M + Opt 13 | Opt14, the final mutant showing |
| | | 14 amino acid substitutions from |
| | | the parent enzyme, and 19 amino |
| | | acid substitutions from the wild |
| | | type enzyme. |
| Thermostability | T101A | |

### Materials and Methods

Overexpression and Purification of PTDH. The buffers used for protein purification included start buffer A (SBA) (0.5 M NaCl, 20% glycerol, and 20 mM Tris, pH 7.6), start buffer B (SBB) (same as A but with 10 mM imidazole) and elute buffer (EB) (0.5 M imidazole, 0.5 M NaCl, 20% glycerol, and 20 mM Tris, pH 7.6). The transformants with pET15b derived vectors were grown in LB medium containing 100 μg/mL ampicillin at 37° C. with good aeration (shaking at 250 RPM). Upon reaching the log phase (OD₆₀₀˜0.6) cells were induced with IPTG (final concentration 0.3 mM) and incubated at 25° C. for 8 h. Cells were harvested by centrifugation at 5,000×g, 4° C., for 15 min and then resuspended in 3 mL/(g cell pellet) start buffer containing 0.6 mg/g lysozyme and stored at −80° C. The frozen cell suspension was thawed at room temperature and lysed by sonication using a Vibra-cell™ sonicator (Newtown, Conn.) with amplitude set at 40%, and with a pulse sequence of 5 s on, 9.9 s off, for about 8-10 min. Cells were centrifuged at 20,000×g at 4° C. for 10 min and the supernatant containing the crude extract was filtered through a 0.45 μm filter to remove any particles. The clarified supernatant was purified by FPLC, with a flow rate of 6 mL/min and fraction size of 8 mL. A POROS MC20 column (7.9 mL bed volume) (Boehringer Mannheim) was charged and equilibrated according to the manufacturer's protocol. The following method was used for purification of PTDH (with His₆-Tag) from a ˜20-60 mL of clarified supernatant (from ˜5-15 g cell paste): 1) load sample through pump, 100 mL, 2) wash column with 100 mL SBB, 3) elute with a linear gradient of 100 mL 100% SBB to 100% EB in 16.7 min, and 4) wash with 100 mL EB. The elute fractions were monitored at λ=280 nm. PTDH (with His₆-Tag) typically eluted from the column halfway through the gradient (40% EB). The protein was concentrated using a Millipore Amicon 8400 stirred ultrafiltration cell with a YM10 membrane at 4° C., washed twice with 75 mL of 50 mM MOPS buffer (pH 7.25 containing 1 mM DTT and 200 mM NaCl) and concentrated again. The enzyme was then stored as concentrated as possible (usually >2 mg/ml) in 200 μL aliquots at −80° C., in a solution of Amicon wash buffer containing 20% glycerol.

Protein Characterization. Protein concentration was determined by the Bradford method using bovine serum albumin as a standard. The purity of the protein was analyzed by SDS-PAGE. SDS-PAGE gels were stained with coomassie brilliant blue. The net pI of the purified mutants and wild type proteins was determined by non-denaturing isoelectric focusing (IEF). The native IEF gel was subsequently activity stained by the same substrate mixture described herein for cell extract activity assay, allowing visualization of the protein by NBT precipitation.

Kinetic Analysis. Initial rates were determined by monitoring the increase in absorbance, corresponding to the production of NAD(P)H (ε_{NAD(P)H}=6.22 mM⁻¹cm⁻¹ at 340 nm). All initial rate assays were carried out at 25° C. using a Varian Cary 100 Bio UV-Visible spectrophotometer. The reaction was initiated by addition of 1.5-3.5 μg of PTDH. Concentrations of NAD⁺ stock solutions were determined by UV-Visible spectroscopy (εNAD⁺=18 mM⁻¹cm⁻¹ at 260 nm). Phosphite concentrations were determined enzymatically by measuring the amount of NADH produced after all phosphite had been oxidized. Michaelis-Menten constants Vₘₐₓ and K_{M} were determined by a series of assays in which five varying concentrations of one substrate were used in the presence of saturating concentrations of the second substrate. The data was then converted to specific activity and fitted with the Michaelis-Menten equation. The WT and double mutants were also analyzed by a sequential matrix of 25 assays. This kinetic data was analyzed with a modified version of Cleland's program. Vₘₐₓ and K_{M} for both phosphite and NAD(P)⁺, were obtained by fitting the data to a sequential ordered mechanism with NAD(P)⁺ binding first, where ν is the initial velocity, V is the maximum velocity, K_{A} and K_{B} are the Michaelis-Menten constants for NAD(P)⁺ and phosphite respectively, A and B are the concentrations of NAD(P)⁺ and phosphite respectively, and Kᵢₐ is the dissociation constant for A (NAD(P)⁺). All assays were performed in duplicate and each series of duplicates was performed a minimum of two times. Data presented in Table 1 represents an average of all statistically relevant data. ν=VAB/(KᵢₐK_{B}+K_{A}B+K_{B}A+AB) (eq. 1)

DNA Sequencing and Analysis. Plasmid DNA was isolated using QIAprep spin plasmid mini-prep kits. Sequencing reactions consisted of 100-200 ng of template DNA, 10 pmol each primer, sequencing buffer and the BigDye reagent. Reactions were carried out for 25 cycles of 96° C. for 30 s, 50° C. for 15 s, 60° C. for 4 min in a PTC-200 Peltier thermal cycler from MJ Research. Prepared samples were submitted to the Biotechnology Center at the University of Illinois for sequencing on an ABI PRISM 3700 sequencer (Applied Biosystems, Foster City, Calif.).

Half-lives of Thermal Inactivation. Purified enzymes (0.2 mg/ml) were incubated in an MJ Research (Watertown, Mass.) PTC-200 thermocylcer to study enzyme inactivation. Timed aliquots were taken at specific time points and placed on ice before assaying. Half-lives of thermal inactivation were calculated using t_{1/2}=ln 2/k_{inact} where k_{inact} is the inactivation rate constant obtained from the slope by plotting log (residual activity/initial activity) versus time. Purified enzymes (0.2 mg/ml) were incubated for 20 min at various fixed elevated temperatures. After incubation, samples were placed on ice for 15 min before being assayed. Residual activity was determined and expressed as a percentage of the initial activity.

Production of PTDH in a bioreactor. PTDH mutant enzymes can be produced in a large-scale bioreactor using standard techniques in microbiological fermentation and downstream processing. For example, a batch reactor containing suitable growth media for bacterial can be operated to grow the bacterial cells (harboring a plasmid that encodes a PTDH enzyme) to appropriate growth density for further downstream processing. Other cultures such as yeast can also be used and other modes of bioreactors such as continuous stirred reactor can also be used to produce and purify the enzyme in a large scale. Appropriate selection markers, oxygen concentration, agitation speeds, nutrient supplements can be optimized using techniques known in the art.

The standard downstream processing steps usually include harvesting cells by continuous centrifugation or cross-flow filtration. For intracellular products, cells are lysed by a French press, mill, sonication, or detergent and the cell debris is removed via crossflow filtration. Crude purification of the protein is generally performed via ammonium sulfate precipitation followed by chromatography (gel permeation, ion exchange, hydrophobic interaction, hydrophilic interaction, and/or metal affinity) and desalting with a dialysis membrane. The purified product is concentrated under vacuum with or without centrifugation and followed by freeze-drying if necessary. Concentration of the protein and activity of the enzyme can be performed using standard assays known to those of ordinary skill in the art.

A membrane bioreactor to evaluate the catalytic performance of the wild type PTDH enzyme, the engineered PTDH variants, and the FDH enzyme, respectively is used. To save time and minimize the variations from reactor setup, a lab-scale enzyme membrane reactor has been purchased from Julich Fine Chemical. In the case of using NAD⁺ as a cofactor, both enzymatic systems are coupled to the production of L-tert-Leucine from trimethyl pyruvate using L-Leucine dehydrogenase. The product formation and substrate depletion is monitored by high-pressure liquid chromatography (HPLC). The total turnover number and stability of each system are determined. Data for the FDH system is consistent with those reported in the literature, which will be used as a benchmark for the development of a proposed phosphite/PtxD system. In the case of using NADP⁺ as a cofactor, the engineered PtxD variants are coupled with recently discovered xylose reductase to convert xylose and glucose into xylitol and sorbitol, respectively. Similarly, the total turnover number and stability of each system will be determined. In both cases, the cofactors are tethered to polyethyleneglycol (PEG, MW=20,000) to increase their sizes as did in the existing FDH-based cofactor regeneration system.

Production of L-tert-Leucine using the PTDH mutants. Small-scale regeneration reactions containing 100 mM ammonium trimethyl pyruvate, 200 mM diammonium phosphite, 0.4 mM NAD, 5.26 U/mL of leucine DH, and 57.5 μg/mL WT PTDH (0.265 U/mL) or round 6 PTDH (0.508 U/mL). The reactions were mixed gently and incubated at 25° C. At fixed time intervals, samples were removed from the reaction and immediately frozen at −80° C. The frozen samples were thawed immediately prior to HPLC analysis. A Shimadzu HPLC equipped with an evaporative light scattering detector was used to quantify the amount of tert-leucine in each sample following separation on a Alltech C-18 prevail column with an isocratic elution of 94.5% water, 4.5% acetonitrile, and 1% acetic acid. The peak area of tert-leucine in each sample was converted to concentration by a standard curve prepared with five known concentrations of authentic L-tert-leucine. The steady state rates for the reactions were determined by fitting the first four data points to a line by linear regression analysis.

T₅₀. Values of T₅₀, the temperature required to reduce initial enzyme activity by 50% after a fixed incubation period, were determined. Briefly, purified enzymes (0.2 mg/mL) were incubated for 20 min at various fixed elevated temperatures. After incubation, samples were placed on ice for 15 min before being assayed using saturating substrate conditions. Residual activity was determined and expressed as a percentage of the initial activity.

Tₒₚₜ. Tₒₚₜ was determined by incubating purified enzymes (0.2 mg/mL) with 1 mM phosphite, 0.5 mM NAD in 50 mM MOPS (pH 7.25) at increasing temperatures for 20 minutes, after which the enzyme activity was determined by monitoring the absorbance increase at 340 nm.

Nucleic acid sequences of the mutants:

Opt12

(SEQ ID NO: 2)

ATGCTGCCGAAACTCGTTATAACTCACCGAGTACACGAAGAGATCCTGCA
ACTGCTGGCGGCACATTGCGAGCTGATAACCAACCAGACCGACAGCACGC
TGACGCGCGAGGAAATTCTGCGCCGCTGTCGCGATGCTCAGGCGATGATG
GCGTTCATGCCCGATCGGGTCGATGCAGACTTTCTTCAAGCCTGCCCTGA
GCTGCGTGTAATCGGCTGCGCGCTCAAGGGCTTCGACAATTTCGATGTGG
ACGCCTGTACTGCCCGCGGGGTCTGGCTGACCTTCGTGCCTGATCTGTTG
ACGGTCCCGACTGCCGAGCTGGCGATCGGACTGGCGGTGGGGCTGGGGCG
GCATCTGCGGGCAGCAGATGCGTTCGTCCGCTCTGGCAAGTTCAAGGGCT
GGCAACCACATTTTTACGGCACGGGGCTGGATAACGCTACGGTCGGCTTC
CTTGGCATGGGCGCCATCGGACTGGCCATGGCTGATCGCTTGCAGGGATG
GGGCGCGACCCTGCAGTACCACGCGGCGAAGGCTCTGGATACACAAACCG
AGCAACGGCTCGGCCTGCGCCAGGTGGCGTGCAGCGAACTCTTCGCCAGC
TCGGACTTCATCCTGCTGGCGCTTCCCTTGAATGCCGATACCCTGCATCT
GGTCAACGCCGAGCTGCTTGCCCTCGTACGGCCGGGCGCTCTGCTTGTAA
ACCCCTGTCGTGGCTCGGTAGTGGATGAAGCCGCCGTGCTCGCGGCGCTT
GAGCGAGGCCAGCTCGGCGGGTATGCGGCGGATGTATTCGAAATGGAAGA
CTGGGCTCGCGCGGACCOGCCOCTGTGCATCGATCCTGCGCTGCTCGCGC
ATCCGAATACGCTGTTCACTCCGCACATAGGGTCGGCAGTGCGCGCGGTG
CGCCTGGAGATTGAACGTTGTGCAGCGCAGAACATCCTCCAGGCATTGGC
AGGTGAGCGCCCAATCAACGCTGTGAACCGTCTGCCCAAGGCCAATCCTG
CCGCAGACTGATAA
Opt 13

(SEQ ID NO: 3)

ATGCTGCCGAAACTCGTTATAACTCACCGAGTACACGAAGAGATCCTGCA
ACTGCTGGCGCCACATTGCGAGCTGATAACCAACCAGACCGACAGCACGC
TGACGCGCGAGGAAATTCTGCGCCGCTGTCGCGATGCTCAGGCGATGATG
GCGTTCATGCCCGATCGGGTCGATGCAGACTTTCTTCAAGCCTGCCCTGA
GCTGCGTGTAATCGGCTGCGCGCTCAAGGGCTTCGACAATTTCGATGTGG
ACGCCTGTACTGCCCGCGGGGTCTGGCTGACCTTCGTGCCTGATCTGTTG
ACGGTCCCGACTGCCGAGCTGGCGATCGGACTGGCGGTGGGGCTGGGGCG
GCATCTGCGGGCAGCAGATGCGTTCGTCCGCTCTGGCAAGTTCAAGGGCT
GGCAACCACATTTCTACGGCACGGGGCTGGATAACTCTACGGTCGGCTTC
CTTGGCATGGGCGCCATCGGACTGGCCATGGCTGATCGCTTGCAGGGATG
GGGCGCGACCCTGCAGTACCACGCGGCGAAGGCTCTGGATACACAAACCG
AGCAACGGCTCGGCCTGCGCCAGGTGGCGTGCAGCGAACTCTTCGCCAGC
TCGGACTTCATCCTGCTGGCGCTTCCCTTGAATGCCGATACCCTGCATCT
GGTCAACGCCGAGCTGCTTGCCCTCGTACGGCCGGGCGCTCTGCTTGTAA
ACCCCTGTCGTGGCTCGGTAGTGGATGAAGCCGCCGTGCTCGCGGCGCTT
GAGCGAGGCCAGCTCGGCGGGTATGCGGCGGATGTATTCGAAATGGAAGA
CTGGGCTCGCGCGGACCGGCCGCTGTGCATCGATCCTGCGCTGCTCGCGC
ATCCGAATACGCTGTTCACTCCGCACATAGGGTCGGCAGTGCGCGCGGTG
CGCCTGGAGATTGAACGTTGTGCAGCGCAGAACATCCTCCAGGCATTGGC
AGGTGAGCGCCCAATCAACGCTGTGAACCGTCTGCCCAAGGCCAATCCTG
CCGCAGACTGATAA
Opt 14

(SEQ ID NO: 4)

ATGCTGCCGAAACTCGTTATAACTCACCGAGTACACGAAGAGATCCTGCA
ACTGCTGGCGCCACATTGCGAGCTGATAACCAACCAGACCGACAGCACGC
TGACGCGCGAGGAAATTCTGCGCCGCTGTCGCGATGCTCAGGCGATGATG
GCGTTCATGCCCGATCGGGTCGATGCAGACTTTCTTCAAGCCTGCCCTGA
GCTGCGTGTAATCGGCTGCGCGCTCAAGGGCTTCGACAATTTCGATGTGG
ACGCCTGTACTGCCCGCGGGGTCTGGCTGACCTTCGTGCCTGATCTGTTG
ACGGTCCCGACTGCCGAGCTGGCGATCGGACTGGCGGTGGGGCTGGGGCG
GCATCTGCGGGCAGCAGATGCGTTCGTCCGCTCTGGCAAGTTCAAGGGCT
GGCAACCACATTTCTACGGCACGGGGCTGGATAACTCTACGGTCGGCTTC
CTTGGCATGGGCGCCATCGGACTGGCCATGGCTGATCGCTTGCAGGGATG
GGGCGCGACCCTGCAGTACCACGCGGCGAAGGCTCTGGATACACAAACCG
AGCAACGGCTCGGCCTGCGCCAGGTGGCGTGCAGCGAACTCATGGCCAGC
TCGGACTTCATCCTGCTGGCGCTTCCCTTGAATGCCGATACCCTGCATCT
GGTCAACGCCGAGCTGCTTGCCCTCGTACGGCCGGGCGCTCTGCTTGTAA
ACCCCTGTCGTGGCTCGGTAGTGGATGAAGCCGCCGTGCTCGCGGCGCTT
GAGCGAGGCCAGCTCGGCGGGTATGCGGCGGATGTATTCGAAATGGAAGA
CTGGGCTCGCGCGGACCGGCCGCTGTGCATCGATCCTGCGCTGCTCGCGC
ATCCGAATACGCTGTTCACTCCGCACATAGGGTCGGCAGTGCGCGCGGTG
CGCCTGGAGATTGAACGTTGTGCAGCGCAGAACATCCTCCAGGCATTGGC
AGGTGAGCGCCCAATCAACGCTGTGAACCGTCTGCCCAAGGCCAATCCTG
CCGCAGACTGATAA.

1. A mutant phosphite dehydrogenase (PTDH) with an increased thermostability and relaxed cofactor specificity for nicotinamade cofactor regeneration as compared to a wild-type phosphite dehydrogenase (PTDH) (SEQ ID NO: 1).

2. The mutant phosphite dehydrogenase of claim 1 comprising a plurality of mutations selected from the group consisting of Q132K, Q137H, R275L, L276C, A146S, F198M, and T101A.

3. The mutant phosphite dehydrogenase of claim 1 comprising a plurality of mutations designated as Q132K, Q137H, R275L, and L276C compared to the wild-type PTDH (SEQ ID NO: 1).

4. The mutant phosphite dehydrogenase of claim 1 comprising a plurality of mutations designated as Q132K, Q137H, R275L, L276C and A146S compared to the wild-type PTDH (SEQ ID NO: 1).

5. The mutant phosphite dehydrogenase of claim 1 comprising a plurality of mutations designated as Q132K, Q137H, R275L, L276C, A146S, and F198M compared to the wild-type PTDH (SEQ ID NO: 1).

6. The mutant phosphite dehydrogenase of claim 1, designated as “Opt12”, comprising a plurality of mutations Q132K, Q137H, R275L, L276C, D13E, M26I, E175A, E332N, C336D, I150F, Q215L, A319E, V315A, V71I, E130K, I313L, and A325V compared to the wild-type PTDH (SEQ ID NO: 1).

7. A The mutant phosphite dehydrogenase of claim 1, designated as “Opt13”, comprising a plurality of mutations Q132K, Q137H, R275L, L276C, A146S, D13E, M26I, E175A, E332N, C336D, I150F, Q215L, A319E, V315A, V71I, E130K, I313L, and A325V compared to the wild-type PTDH (SEQ ID NO: 1).

8. The mutant phosphite dehydrogenase of claim 1, designated as “Opt14”, comprising a plurality of mutations Q132K, Q137H, R275L, L276C, A146S, F198M, D13E, M26I, E175A, E332N, C336D, I150F, Q215L, A319E, V315A, V711, E130K, I313L, and A325V compared to the wild-type PTDH (SEQ ID NO: 1).

9. A mutant phosphite dehydrogenase (PTDH) (“Opt14”) consisting essentially of mutations designated as Q132K, Q137H, R275L, L276C, A146S, F198M, D13E, M26I, E175A, E332N, C336D, I150F, Q215L, A319E, V315A, V71I, E130K, I313L, and A325V compared to the wild-type PTDH (SEQ ID NO: 1).

10. The mutant phosphite dehydrogenase mutant of claim 1 further comprising an amino acid mutation designated A176R.

11. A nucleic acid molecule encoding any one of the phosphite dehydrogenase mutants of claims 1-9.

12. A nucleic acid molecule encoding a mutant phosphite dehydrogenase, the nucleic acid molecule comprising a sequence selected from the group consisting of SEQ ID NO: 2 (“Opt12”), SEQ ID NO: 3 (“Opt13”), and SEQ ID NO: 4 (“Opt14”).

13. A phosphite dehydrogenase mutant of any one of claims 1-9 is substantially purified.

14. A phosphite dehydrogenase mutant of any one of claims 1-9 is heterologously expressed.

15. A phosphite dehydrogenase mutant of any one of claims 1-9 is recombinant.

16. A host cell transformed with the nucleic acid molecule of claim 11 or 12.

17. An expression vector encoding the nucleic acid molecule of claim 11 or 12.

18. A method of generating at least one of NADH and NADPH, comprising:
(a) providing a mutant phosphite dehydrogenase, wherein the mutant has an amino acid mutation selected from the group consisting of mutations Q132K, Q137H, R275L, L276C, A146S, F198M, and T101A as compared to the wild-type and;
(b) generating at least one of NADH and NADPH by a reduction reaction of at least one of NAD⁺ and NADP⁺.

19. The method of claim 18, wherein the mutant phosphite dehydrogenase is designated as one of Opt12 or Opt13 or Opt14 as in claim 6 or 7 or 8 respectively.

20. Use of the phosphite dehydrogenase of one of claims 6-8 to regenerate one of NAD⁺, NADP⁺ or both NAD⁺ and NADP⁺.


SEQUENCE LISTING (7 sequences)

SEQ ID NO: 1 - protein, 336 aa, Pseudomonas stutzeri
MLPKLVITHRVHDEILQLLAPHCELMTNQTDSTLTREEILRRCRDAQAMMAFMPDRVDADFLQACPELRVVGCALKGFDNFDVDACTARGVWLTFVPDLLTVPTAELAIGLAVGLGRHLRAADAFVRSGEFQGWQPQFYGTGLDNATVGILGMGAIGLAMADRLQGWGATLQYHEAKALDTQTEQRLGLRQVACSELFASSDFILLALPLNADTQHLVNAELLALVRPGALLVNPCRGSVVDEAAVLAALERGQLGGYAADVFEMEDWARADRPRLIDPALLAHPNTLFTPHIGSAVRAVRLEIERCAAQNIIQVLAGARPINAANRLPKAEPAAC

SEQ ID NO: 2 - DNA, 1014 bp, Artificial Sequence
  Description of Artificial Sequence Synthetic polynucleotide
atgctgccgaaactcgttataactcaccgagtacacgaagagatcctgcaactgctggcgccacattgcgagctgataaccaaccagaccgacagcacgctgacgcgcgaggaaattctgcgccgctgtcgcgatgctcaggcgatgatggcgttcatgcccgatcgggtcgatgcagactttcttcaagcctgccctgagctgcgtgtaatcggctgcgcgctcaagggcttcgacaatttcgatgtggacgcctgtactgcccgcggggtctggctgaccttcgtgcctgatctgttgacggtcccgactgccgagctggcgatcggactggcggtggggctggggcggcatctgcgggcagcagatgcgttcgtccgctctggcaagttcaagggctggcaaccacatttttacggcacggggctggataacgctacggtcggcttccttggcatgggcgccatcggactggccatggctgatcgcttgcagggatggggcgcgaccctgcagtaccacgcggcgaaggctctggatacacaaaccgagcaacggctcggcctgcgccaggtggcgtgcagcgaactcttcgccagctcggacttcatcctgctggcgcttcccttgaatgccgataccctgcatctggtcaacgccgagctgcttgccctcgtacggccgggcgctctgcttgtaaacccctgtcgtggctcggtagtggatgaagccgccgtgctcgcggcgcttgagcgaggccagctcggcgggtatgcggcggatgtattcgaaatggaagactgggctcgcgcggaccggccgctgtgcatcgatcctgcgctgctcgcgcatccgaatacgctgttcactccgcacatagggtcggcagtgcgcgcggtgcgcctggagattgaacgttgtgcagcgcagaacatcctccaggcattggcaggtgagcgcccaatcaacgctgtgaaccgtctgcccaaggccaatcctgccgcagactgataa

SEQ ID NO: 3 - DNA, 1014 bp, Artificial Sequence
  Description of Artificial Sequence Synthetic polynucleotide
atgctgccgaaactcgttataactcaccgagtacacgaagagatcctgcaactgctggcgccacattgcgagctgataaccaaccagaccgacagcacgctgacgcgcgaggaaattctgcgccgctgtcgcgatgctcaggcgatgatggcgttcatgcccgatcgggtcgatgcagactttcttcaagcctgccctgagctgcgtgtaatcggctgcgcgctcaagggcttcgacaatttcgatgtggacgcctgtactgcccgcggggtctggctgaccttcgtgcctgatctgttgacggtcccgactgccgagctggcgatcggactggcggtggggctggggcggcatctgcgggcagcagatgcgttcgtccgctctggcaagttcaagggctggcaaccacatttctacggcacggggctggataactctacggtcggcttccttggcatgggcgccatcggactggccatggctgatcgcttgcagggatggggcgcgaccctgcagtaccacgcggcgaaggctctggatacacaaaccgagcaacggctcggcctgcgccaggtggcgtgcagcgaactcttcgccagctcggacttcatcctgctggcgcttcccttgaatgccgataccctgcatctggtcaacgccgagctgcttgccctcgtacggccgggcgctctgcttgtaaacccctgtcgtggctcggtagtggatgaagccgccgtgctcgcggcgcttgagcgaggccagctcggcgggtatgcggcggatgtattcgaaatggaagactgggctcgcgcggaccggccgctgtgcatcgatcctgcgctgctcgcgcatccgaatacgctgttcactccgcacatagggtcggcagtgcgcgcggtgcgcctggagattgaacgttgtgcagcgcagaacatcctccaggcattggcaggtgagcgcccaatcaacgctgtgaaccgtctgcccaaggccaatcctgccgcagactgataa

SEQ ID NO: 4 - DNA, 1014 bp, Artificial Sequence
  Description of Artificial Sequence Synthetic polynucleotide
atgctgccgaaactcgttataactcaccgagtacacgaagagatcctgcaactgctggcgccacattgcgagctgataaccaaccagaccgacagcacgctgacgcgcgaggaaattctgcgccgctgtcgcgatgctcaggcgatgatggcgttcatgcccgatcgggtcgatgcagactttcttcaagcctgccctgagctgcgtgtaatcggctgcgcgctcaagggcttcgacaatttcgatgtggacgcctgtactgcccgcggggtctggctgaccttcgtgcctgatctgttgacggtcccgactgccgagctggcgatcggactggcggtggggctggggcggcatctgcgggcagcagatgcgttcgtccgctctggcaagttcaagggctggcaaccacatttctacggcacggggctggataactctacggtcggcttccttggcatgggcgccatcggactggccatggctgatcgcttgcagggatggggcgcgaccctgcagtaccacgcggcgaaggctctggatacacaaaccgagcaacggctcggcctgcgccaggtggcgtgcagcgaactcatggccagctcggacttcatcctgctggcgcttcccttgaatgccgataccctgcatctggtcaacgccgagctgcttgccctcgtacggccgggcgctctgcttgtaaacccctgtcgtggctcggtagtggatgaagccgccgtgctcgcggcgcttgagcgaggccagctcggcgggtatgcggcggatgtattcgaaatggaagactgggctcgcgcggaccggccgctgtgcatcgatcctgcgctgctcgcgcatccgaatacgctgttcactccgcacatagggtcggcagtgcgcgcggtgcgcctggagattgaacgttgtgcagcgcagaacatcctccaggcattggcaggtgagcgcccaatcaacgctgtgaaccgtctgcccaaggccaatcctgccgcagactgataa

SEQ ID NO: 5 - DNA, 24 bp, Artificial Sequence
  Description of Artificial Sequence Synthetic primer
tttttggatggaggaattcatatg

SEQ ID NO: 6 - DNA, 34 bp, Artificial Sequence
  Description of Artificial Sequence Synthetic primer
gtacgtcgatacatgtttatcagtctgcggcagg

SEQ ID NO: 7 - protein, 6 aa, Artificial Sequence
  Description of Artificial Sequence Synthetic 6xHis tag
HHHHHH